(19) United States
(12) Patent Application Publication
(10) Pub. No.: US 2023/0201486 A1
(43) Pub. Date: Jun. 29, 2023

(54) ULTRASONIC VAPORIZATION ASSEMBLY AND ULTRASONIC VAPORIZATION DEVICE

(71) Applicants: Ming TANG, Shenzhen (CN); Yongxiong ZHU, Shenzhen (CN); Guanglin OUYANG, Shenzhen (CN)

(72) Inventors: Ming TANG, Shenzhen (CN); Yongxiong ZHU, Shenzhen (CN); Guanglin OUYANG, Shenzhen (CN)

(73) Assignee: Shenzhen Moore Vaporization Health & Medical Technology Co., Ltd., Shenzhen (CN)

(21) Appl. No.: 18/081,209

(22) Filed: Dec. 14, 2022

(30) Foreign Application Priority Data
Dec. 29, 2021 (CN) 202111642024.3

(51) Int. Cl. A61M 11/00

(52) U.S. Cl. CPC A61M 11/005

(57) ABSTRACT

An ultrasonic vaporization assembly includes: a piezoelectric drive element provided with a first through hole; a vaporizer plate stacked with the piezoelectric drive element, the vaporizer plate having a microporous zone at a position corresponding to the first through hole; and a loading board arranged on a side surface of the vaporizer plate which faces away from the piezoelectric drive element, the loading board being provided with a second through hole at a position corresponding to the microporous zone. A ratio of a pore size of the second through hole to a diameter of the microporous zone is greater than or equal to 0.75 and less than or equal to 1.5.

### CROSS-REFERENCE TO PRIOR APPLICATION

Priority is claimed to Chinese Patent Application No. 202111642024.3, filed on Dec. 29, 2021, the entire disclosure of which is hereby incorporated by reference herein.

### FIELD

The present invention relates to the technological field of the ultrasonic vaporization, and in particular, to an ultrasonic vaporization assembly and an ultrasonic vaporization device.

### BACKGROUND

With the development of medical technology, an aerosol inhalation administration method has been accepted by more and more people due to advantages of low damage and high efficacy. Ultrasonic microporous vaporization administration is a rapidly developing administration method.

An existing ultrasonic microporous vaporization assembly is mainly composed of a piezoelectric ceramic ring and a microporous vaporizer plate. A main working principle is to use inverse piezoelectric effect of the piezoelectric ceramic ring to generate high frequency micro-oscillation, so as to split a surface of an aerosol-forming substrate. Thus, aerosol of tiny particles with vector property is formed, and then disperses in the form of mist.

However, the existing ultrasonic microporous vaporization assembly has large invalid vibration loss, high working noise and serious heating problems; Moreover, the small-particle-size aerosol accounts for a low proportion in the vaporized aerosol.

### SUMMARY

In an embodiment, the present invention provides an ultrasonic vaporization assembly, comprising: a piezoelectric drive element provided with a first through hole; a vaporizer plate stacked with the piezoelectric drive element, the vaporizer plate comprising a microporous zone at a position corresponding to the first through hole; and a loading board arranged on a side surface of the vaporizer plate which faces away from the piezoelectric drive element, the loading board being provided with a second through hole at a position corresponding to the microporous zone, wherein a ratio of a pore size of the second through hole to a diameter of the microporous zone is greater than or equal to 0.75 and less than or equal to 1.5.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of an ultrasonic vaporization device according to an embodiment of this application;

FIG. 2 is a structural exploded view of an ultrasonic vaporization assembly according to an embodiment of this application;

FIG. 3 is a vertical sectional view of a vaporizer plate according to an embodiment of this application;

FIG. 4 is a side sectional view of a first embodiment of an ultrasonic vaporization assembly with a boss of this application;

FIG. 5 is a side sectional view of a second embodiment of an ultrasonic vaporization assembly with a boss of this application;

FIG. 6 is a side sectional view of a first embodiment of an ultrasonic vaporization assembly without a boss of this application;

FIG. 7 is a side sectional view of a second embodiment of an ultrasonic vaporization assembly without a boss of this application; and

FIG. 8 is a schematic structural diagram of a loading board of this application.

### DETAILED DESCRIPTION

In an embodiment, the present invention provides an ultrasonic vaporization assembly and an ultrasonic vaporization device aimed to solve problems that the existing ultrasonic vaporization assembly has great invalid vibration loss, high working noise, serious heat generation, and relatively low proportion of small-particle-size aerosol in vaporized aerosol.

In an embodiment, the present invention provides an ultrasonic vaporization assembly. The ultrasonic vaporization assembly includes a piezoelectric drive element, a vaporizer plate, and a loading board, where the piezoelectric drive element is provided with a first through hole; the vaporizer plate is stacked with the piezoelectric drive element, and the vaporizer plate is provided with a microporous zone at a position corresponding to the first through hole; the loading board is arranged on a side surface of the vaporizer plate which faces away from the piezoelectric drive element; and the loading board is provided with a second through hole at a position corresponding to the microporous zone, where a ratio of a pore size of the second through hole to a diameter of the microporous zone is greater than or equal to 0.75 and less than or equal to 1.5.

The ratio of the diameter of the microporous zone to the pore size of the second through hole is 1.

The microporous zone is planar; or, the microporous zone raises towards a mist outlet direction of the ultrasonic vaporization assembly to form a boss.

The microporous zone is located in a central area of the vaporizer plate.

The microporous zone includes a plurality of vaporization through holes, and the pore size of the vaporization through hole gradually decreases along the mist outlet direction of the ultrasonic vaporization assembly.

The loading board is a metal sheet, and a thickness of the metal sheet ranges from 0.4 mm to 0.7 mm.

A ratio of an outer diameter of the vaporizer plate to the thickness of the loading board is between 23 and 40.

The vaporizer plate has a liquid inlet surface and a mist outlet surface that are arranged opposite to each other; and the loading board is arranged on the liquid inlet surface, and the piezoelectric drive element is arranged on the mist outlet surface; or the vaporizer plate has a liquid inlet surface and a mist outlet surface that are arranged opposite to each other; and the loading board is arranged on the mist outlet surface, and the piezoelectric drive element is arranged on the liquid inlet surface.

The piezoelectric drive element, the vaporizer plate, and the loading board are all circular and have the same outer diameter; the vaporizer plate is a metallic screen mesh; and the piezoelectric drive element is a piezoelectric ceramic ring.

In order to solve the above technical problems, another technical solution adopted by this application is to provide an ultrasonic vaporization device. The ultrasonic vaporization device includes a housing, an ultrasonic vaporization assembly, and a power supply assembly, where the housing has a liquid storage cavity and a liquid outlet; the ultrasonic vaporization assembly is arranged at the liquid outlet, configured to vaporize an aerosol-forming substrate of the liquid storage cavity when power is turned on; the ultrasonic vaporization assembly is the above-mentioned ultrasonic vaporization assembly; and the power supply assembly is electrically connected to the ultrasonic vaporization assembly, configured to supply power to the ultrasonic vaporization assembly.

The embodiments of this application has the following beneficial effect: Different from the prior art, the embodiments of this application provide an ultrasonic vaporization assembly and an ultrasonic vaporization device; in the ultrasonic vaporization assembly, a loading board is added and placed on a side surface of a vaporizer plate away from an piezoelectric drive element, which effectively improves overall structural rigidity of the vaporizer plate, so that a vibration mode of the vaporizer plate is effectively restricted, thus not only reducing invalid vibration of the vaporizer plate and vibration loss caused by the invalid vibration, but also greatly reducing noise caused by the invalid vibration of the vaporizer plate and a temperature rise of the vaporizer plate; meanwhile, the proportion of small-size particles in vaporized particles is effectively increased. Besides, a ratio of a pore size of a second through hole to a diameter of a microporous zone is greater than or equal to 0.75 and less than or equal to 1.5, so that the ultrasonic vaporization assembly achieves a desired noise reduction effect and has less temperature rise. In addition, the added loading board can reduce a clamping force of a housing and the piezoelectric drive element on the vaporizer plate after installation of the ultrasonic vaporization assembly and the housing, thus effectively reducing vibration performance attenuation caused by the clamping of the vaporizer plate. Moreover, the second through hole is provided at a position of the loading board corresponding to the microporous zone, to effectively ensure that a liquid inlet surface or a mist outlet surface of the vaporizer plate is in communication with the external, so as to ensure that an aerosol substrate or vaporized aerosol particles can pass through smoothly.

The technical solutions in the embodiments of this application will be clearly and completely described below in combination with the accompanying drawings of the embodiments of this application. Obviously, the described embodiments are only part of the embodiments of this application but not all embodiments. Based on the embodiments of this application, all other embodiments obtained by persons of ordinary skill in the art without creative efforts fall within the protection scope of this application.

The terms “first”, “second” and “third” in this application are only used for description and cannot be understood as indicating or implying relative importance or implying the number of an indicated technical feature. Therefore, the feature defined with “first”, “second”, and “third” may expressly or implicitly include at least one of the features. In the description of this application, “many” means at least two, such as two, three and the like, unless otherwise explicitly and specifically defined. All directional indications (such as up, down, left, right, front, back...) in the embodiments of this application are only used to explain relative position relations, movement situation and the like between components under a certain posture (as shown in the accompanying drawings). If the certain posture changes, the directional indication also changes accordingly. Besides, the terms “include” and “have” and any variations thereof are intended to cover non-exclusive inclusion. For example, a process, method, system, product or device including a series of steps or units is not limited to the listed steps or units, but optionally also includes unlisted steps or units, or optionally also includes other steps or units inherent to the process, method, product or device.

The “embodiment” mentioned herein means that a specific feature, structure, or characteristic described in combination with the embodiments may be included in at least one embodiment of this application. The appearances of the phrase in various positions of the specification do not necessarily refer to the same embodiment, nor a separate or an alternative embodiment that is mutually exclusive with other embodiments. Persons skilled in the art can explicitly and implicitly understand that the embodiments described herein may be combined with other embodiments.

This application is described in detail below in combination with the accompanying drawings and the embodiments.

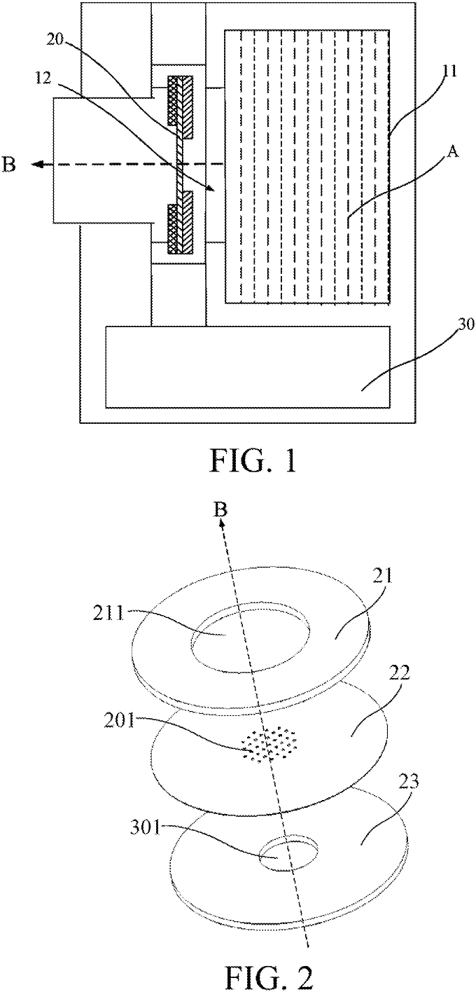

Referring to FIG. 1, FIG. 1 is a schematic structural diagram of an ultrasonic vaporization device according to an embodiment of this application; In the embodiment, an ultrasonic vaporization device is provided, where the ultrasonic vaporization device can be used in the technical field of medical treatment, beauty, electronic cigarette and the like. The structure of the ultrasonic vaporization device includes a housing 10, an ultrasonic vaporization assembly 20, and a power supply assembly 30.

The housing 10 has a liquid storage cavity 11 and a liquid outlet 12 connected with the liquid storage cavity 11. The liquid storage cavity 11 is used for containing an aerosol-forming substrate A. The aerosol-forming substrate A is a liquid medicine formed by a certain medicine dispersed in a liquid solvent, e-liquid or any other liquid suitable for ultrasonic atomization. The ultrasonic vaporization assembly 20 is arranged at the liquid outlet 12 of the housing 10, configured to generate high-frequency oscillation when power is turned on, so as to atomize the aerosol-forming substrate A. The power supply assembly 30 is electrically connected to the ultrasonic vaporization assembly 20, configured to supply power to the ultrasonic vaporization assembly 20. The power supply assembly 30 may specifically be a lithium-ion battery.

The existing ultrasonic vaporization assembly is generally composed of a piezoelectric ceramic ring and a vaporizer plate. However, when the existing ultrasonic vaporization assembly is working, vibration mode constraint is insufficient and the vibration is not concentrated, which leads to high working noise, serious heating and great invalid vibration loss. In addition, the existing vaporizer plate generally has a boss structure. After stamping the boss, the vaporizer plate with the boss structure tends to enlarge micro-pores on the vaporizer plate, resulting in a larger atomizing particle size of the vaporizer plate, which reduces the proportion of small-particle-size aerosol in the vaporized aerosol and is not beneficial to vaporization inhalation.

The embodiments of this application provide an ultrasonic vaporization assembly 20, where the ultrasonic vaporization assembly 20 can effectively constrain the ultrasonic vibration mode of the vaporizer plate 22, so that the vaporizer plate 22 can vibrate in a concentrated manner, which effectively reduces the invalid vibration of the vaporizer plate 22, the working noise of the ultrasonic vaporization assembly 20, and the temperature rise. In this way, the vaporization amount per unit time of the ultrasonic vaporization assembly 20 is increased. Furthermore, the ultrasonic vaporization assembly 20 can greatly improve the proportion of the small-particle-size aerosol in the aerosol particles formed by the vaporization.

Referring to FIG. 2, FIG. 2 is a structural exploded view of an ultrasonic vaporization assembly according to an embodiment of this application; the ultrasonic vaporization assembly 20 includes a piezoelectric drive element 21, a vaporizer plate 22, and a loading board 23 that are stacked in sequence.

The piezoelectric drive element 21 is configured to convert electrical energy into mechanical energy by using an inverse piezoelectric effect when power is turned on, so as to generate high frequency vibration. The piezoelectric drive element 21 is provided with a first through hole 211, and the aerosol-forming substrate A can enter the surface of the vaporizer plate 22 through the first through hole 211; or the aerosol particles, formed after the aerosol-forming substrate A is vaporized by the vaporizer plate 22, are scattered through the first through hole 211. In the specific embodiments, the piezoelectric drive element 21 may be the piezoelectric ceramic ring. A cross section of the piezoelectric drive element 21 may be in the shape of a circle, a ring, a square and the like, which may be selected according to an actual situation.

The vaporizer plate 22 has a liquid inlet surface 221 and a mist outlet surface 222 that are opposite to each other. The piezoelectric drive element 21 is stacked on the liquid inlet surface 221 or the mist outlet surface 222 of the vaporizer plate 22. The vaporizer plate 22 has a microporous zone 201, where the microporous zone 201 is used to break up the aerosol-forming substrate A into small particles under the driving of the piezoelectric drive element 21, and the small particles escape through the microporous zone 201, so as to form the aerosol. The aerosol-generating substrate enters the microporous zone 201 from the liquid inlet surface 221 of the vaporizer plate 22, and the scattered small particles escape from the microporous zone 201 from the mist outlet surface 222 of the vaporizer plate 22. Specifically, the vaporizer plate 22 may be a screen mesh; the vaporizer plate 22 may be made of materials such as stainless steel, titanium alloy, palladium alloy and the like, which is not limited in this application. The vaporizer plate 22 may be a vaporizer plate 22a/22b in the following embodiments.

Specifically, the microporous zone 201 of the vaporizer plate 22 is arranged corresponding to the first through hole 211, and a pore size of the first through hole 211 may be greater than or equal to a diameter of the microporous zone 201, so as to ensure that all vaporized particles of the aerosol-forming substrate A can pass through. Certainly, the pore size of the first through hole 211 may also be slightly smaller than the diameter of the microporous zone 201, which is not limited in this application. The vaporizer plate 22 and the piezoelectric drive element 21 specifically may be connected together by gluing, spot welding, or the like.

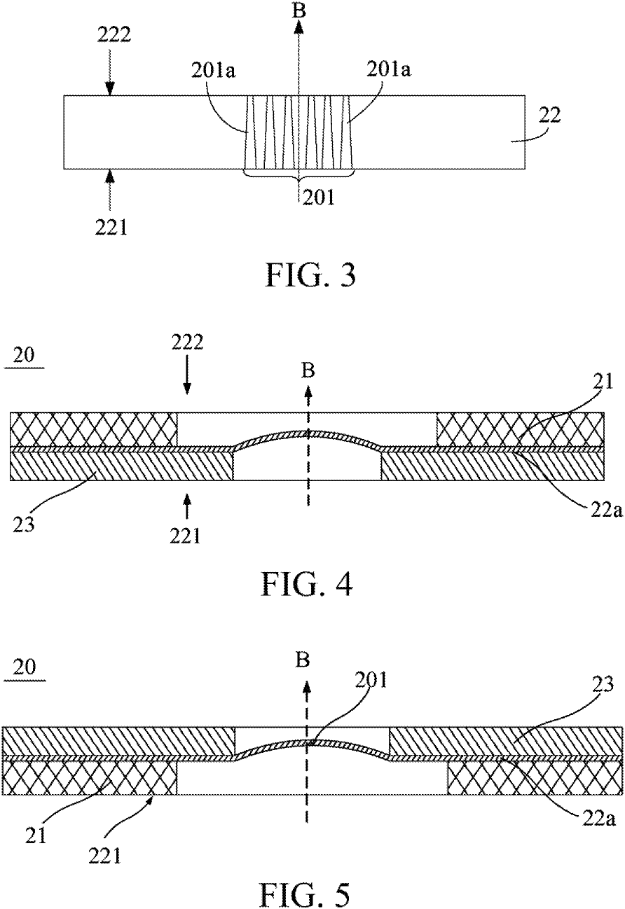

Specifically, referring to FIG. 2 and FIG. 3, FIG. 3 is a vertical sectional view of a vaporizer plate according to an embodiment of this application. The microporous zone 201 of the vaporizer plate 22 includes a plurality of vaporization through holes 201a. The pore size of each vaporization through hole 201a gradually decreases along a mist outlet direction B of the ultrasonic vaporization assembly 20. In this way, the vaporization through hole 201a can be used to reduce the particle size of the vaporized aerosol, so as to improve the proportion of the small-particle-size aerosol in the vaporized aerosol, and avoid the problem that an increase in the pore size of the vaporization through hole 201a due to the stamping causes a decrease in the proportion of the small-particle-size aerosol in the vaporized aerosol can be avoided.

In a specific embodiment, referring to FIG. 4 or FIG. 5, the microporous zone 201 of the vaporizer plate 22a raises towards the mist outlet direction B of the ultrasonic vaporization assembly 20 to form the boss. In this embodiment, referring to Table 1, compared with the ultrasonic vaporization assembly 20a (sample 2) corresponding to the microporous zone 201 of the vaporizer plate 22a without the boss, in the case of the same diameter of the microporous zone 201, the vaporizer plate 22a with the boss structure provided by this embodiment can increase a surface area of the microporous zone 201 to a certain extent, which means increasing a contact area with the aerosol-forming substrate A, thereby effectively improving the vaporization amount per unit time of the ultrasonic vaporization assembly 20a (sample 4). Table 1 shows corresponding experimental data under the condition that 5 ml of aerosol-forming substrate is vaporized and the specifications of the vaporizer plate 22a/22b and the piezoelectric drive element 21 are the same.

Table 1 is the experimental comparison data between the ultrasonic vaporization assembly of this application and the existing ultrasonic vaporization assembly

| Serial number | Whether there is a piezoelectric drive element | Whether a vaporizer plate has a boss | Whether there is a loading board | Average vaporization rate | Average proportion of small-size particles (%) | Noise | Temperature increment (/°C) |
|---|---|---|---|---|---|---|---|
| Sample 1 | Yes | No | No | 3 | 78 | High | 17 |
| Sample 2 | Yes | No | Yes | 7.5 | 82 | Barely audible | 8 |
| Sample 3 | Yes | Yes | No | 4.5 | 63 | High | 17 |
| Sample 4 | Yes | Yes | Yes | 8.1 | 80 | Barely audible | 8 |

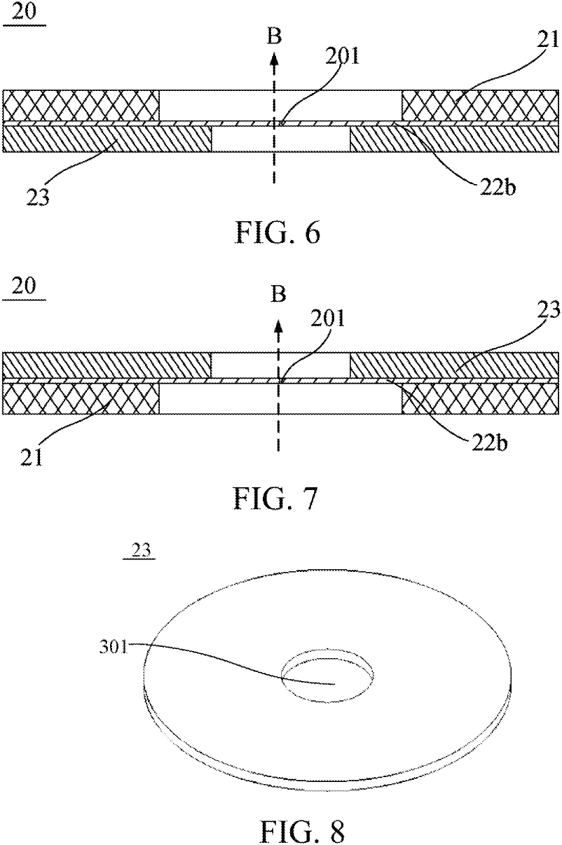

In another specific embodiment, referring to FIG. 6 or FIG. 7, the microporous zone 201 of the vaporizer plate 22b is planar. Compared with the ultrasonic vaporization assembly 20 (sample 4) corresponding to the boss formed in the microporous zone 201 of the vaporizer plate 22b, the ultrasonic vaporization assembly 20 (sample 2) corresponding to this specific embodiment can avoid the problem of an increase in the particle size of the vaporized aerosol due to an enlarged vaporization through hole 201a on the vaporizer plate 22b after the boss is stamped, thus further improving the proportion of the small-particle-size aerosol in the vaporized aerosol.

Referring to FIG. 2 and FIG. 8, FIG. 8 is a schematic structural diagram of a loading board of this application. The loading board 23 is arranged on a side surface of the vaporizer plate 22a/22b which faces away from the piezoelectric drive element 21; the loading board 23 specifically may be closely connected to the vaporizer plate 22a/22b by gluing, spot welding, or the like.

Referring to Table 1, the added loading board 23 effectively improves overall structural rigidity of the vaporizer plate 22a/22b, changes driving frequency of the vaporizer plate 22a/22b, and effectively constrains the vibration mode of the vaporizer plate 22a/22b. Besides, the added loading board 23 can constrain the vibration mode of the vaporizer plate 22a/22b to a central area of the vaporizer plate 22a/22b, thereby not only reducing the invalid vibration of the vaporizer plate 22a/22b, but also greatly reducing the noise caused by the invalid vibration of the vaporizer plate 22a/22b and the temperature rise of the vaporizer plate 22a/22b. Meanwhile, referring to Table 1, the added loading board 23 effectively increases the vaporization amount of the aerosol-forming substrate A per unit time, and the proportion of the small-particle-size aerosol in the vaporized aerosol. In addition, referring to Table 1, on the basis of adding the loading board 23, the microporous zone 201 of the vaporizer plate 22b is made planar, which can further increase the proportion of the small-particle-size aerosol in the vaporized aerosol. Furthermore, air bubbles generated during working of the ultrasonic vaporization assembly 20 can be reduced.

Specifically, the loading board 23 is provided with a second through hole 301, where the second through hole 301 is arranged corresponding to the microporous zone 201 of the vaporizer plate 22a/22b, so that the microporous zone 201 of the vaporizer plate 22a/22b is at least partially exposed, thereby ensuring that the aerosol-forming substrate A or the vaporized aerosol particles can pass through smoothly.

In a specific embodiment, a ratio of a pore size of the second through hole 301 of the loading board 23 to a diameter of the microporous zone 201 of the vaporizer plate 22 is greater than or equal to 0.75 and less than or equal to 1.5; Within the ratio range, the vibration mode of the vaporizer plate 22 can be effectively constrained, so that the vibration can be concentrated, thereby reducing the invalid vibration, the vibration loss, and the working noise and the temperature rise of the ultrasonic vaporization assembly 20. In a specific embodiment, the ratio of the pore size of the second through hole 301 to the diameter of the microporous zone 201 is 1. Under this solution, the ultrasonic vaporization assembly 20 can achieve the best noise reduction effect, and the temperature rise of the ultrasonic vaporization 20 is the lowest.

The shape of the loading board 23, the piezoelectric drive element 21 and/or the vaporizer plate 22a/22b may be circular; the outer diameter of the loading board 23, the piezoelectric drive element 21 and/or the vaporizer plate 22a/22b may also be the same, so as to facilitate subsequent installation and use. Specifically, the material of the loading board 23 may be the same as the material of the vaporizer plate 22a/22b, and specifically may be a rigid metal material such as stainless steel, titanium alloy, palladium alloy and the like, so as to enhance the strength of the ultrasonic vaporization assembly 20, and better constrain the vibration mode.

In a specific embodiment, the ratio of the outer diameter of the vaporizer plate 22a/22b to the thickness of the loading board 23 may be between 23 and 40; In this range, a desired effect of reducing the working noise and temperature rise of the ultrasonic vaporization assembly 20 can be achieved, and the vibration loss caused by the invalid vibration can be effectively decreased. Specifically, when the outer diameter of the vaporizer plate 22 is 10 mm, the thickness of the loading board 23 may range from 0.25 mm to 0.43 mm. in this case, the ultrasonic vaporization assembly 20 can achieve a desired effect of reducing the working noise and temperature rise, and can effectively decrease the vibration loss caused by the invalid vibration. When the outer diameter of the vaporizer plate 22 is 16 mm, the thickness of the loading board 23 may range from 0.4 mm to 0.7 mm. In this case, the ultrasonic vaporization assembly 20 can achieve the best effect of reducing the noise and temperature rise.

Referring to FIG. 4, FIG. 4 is a side sectional view of a first embodiment of an ultrasonic vaporization assembly with a boss of this application. In a specific embodiment, the microporous zone 201 of the vaporizer plate 22a raises towards the mist outlet direction B of the ultrasonic vaporization assembly 20 to form the boss. The carrier plate 23 is disposed on the liquid inlet surface 221 of the atomizing sheet 22a and is closely combined with the atomizing sheet 22a, and the piezoelectric driving element 21 is disposed on the mist outlet surface 222 of the atomizing sheet 22a and is closely combined with the atomizing sheet 22a.

Referring to FIG. 5, FIG. 5 is a side sectional view of a second embodiment of an ultrasonic vaporization assembly with a boss of this application. In another specific embodiment, the microporous zone 201 of the vaporizer plate 22a raises towards the mist outlet direction B of the ultrasonic vaporization assembly 20 to form the boss. The loading board 23 is arranged on the mist outlet surface 222 of the vaporizer plate 22a and is closely connected to the vaporizer plate 22a, and the piezoelectric drive element 21 is arranged on the liquid inlet surface 221 of the vaporizer plate 22a and is closely connected to the vaporizer plate 22a.

In the above specific embodiment, referring to Table 1, the vaporizer plate 22a has a boss structure; In the case of the same diameter of the microporous zone 201, the vaporizer plate 22a with the boss structure can increase the surface area of the microporous zone 201 to a certain extent, which means increasing the contact area with the aerosol-forming substrate A, thereby improving the vaporization amount per unit time of the ultrasonic vaporization assembly 20 to a certain extent. Specifically, the average vaporization rate of the vaporizer plate 22a with the boss can reach 8.1 mg/s.

Referring to FIG. 6, FIG. 6 is a side sectional view of a first embodiment of an ultrasonic vaporization assembly without a boss of this application. In another embodiment, the microporous zone 201 of the vaporizer plate 22b is planar. The loading board 23 is arranged on the side of the liquid inlet surface 221 of the vaporizer plate 22b and is closely connected to the vaporizer plate 22b, and the piezoelectric drive element 21 is arranged on the side of the mist outlet surface 222 of the vaporizer plate 22b and is closely connected to the vaporizer plate 22b.

Referring to FIG. 7, different from FIG. 6, the loading board 23 is arranged on the side of the mist outlet surface 222 of the vaporizer plate 22a and is closely connected to the vaporizer plate 22b, and the piezoelectric drive element 21 is arranged on the side of the liquid inlet surface 221 of the vaporizer plate 22b and is closely connected to the vaporizer plate 22b. That is, in FIG. 6 and FIG. 7, the position of the loading board 23 and the position of the piezoelectric drive element 21 are different. In practical application, changing the installation position of the piezoelectric drive element 21 on the vaporizer plate 22a/22b can effectively widen the application range of the ultrasonic vaporization assembly 20.

In the above specific embodiment, referring to Table 1, the vaporizer plate 22b is the planar vaporizer plate without the boss. The structure can avoid problem of that the vaporization particle size of the vaporizer plate increases due to enlarged micro-pores on the vaporizer plate after the boss is stamped, and thus the proportion of small-size particles in the vaporized particles. Specifically, in the above embodiment, the average proportion of small-size particles in the vaporized particles of the vaporizer plate 22b can reach 82%, which further improves the vaporization effect. In this embodiment, in a process of vaporizing 5 ml of aerosol-forming substrate A, the temperature rise of the ultrasonic vaporization assembly 20 with the non-boss vaporizer plate 22b is about 8 degrees Celsius, and the working noise is also significantly reduced. During the working process, the average vaporization rate of the ultrasonic vaporization assembly 20 is 7.5 mg/s, while the average vaporization rate of the conventional ultrasonic vaporization assembly is 3 mg/s. Therefore, compared with the conventional vaporizer plate, the working efficiency of the ultrasonic vaporization assembly 20 is also greatly improved.

In the ultrasonic vaporization assembly 20 provided by the embodiments of this application, a loading board 23 is added and placed on a side surface of a vaporizer plate 22a/22b away from a piezoelectric drive element 21, which effectively improves overall structural rigidity of the vaporizer plate 22a/22b, so that a vibration mode of the vaporizer plate 22a/22b is effectively constrained, thus not only decreasing invalid vibration of the vaporizer plate 22a/22b, but also greatly reducing noise caused by the invalid vibration of the vaporizer plate 22a/22b and a temperature rise of the vaporizer plate 22a/22b; meanwhile, the proportion of small-size particles in vaporized particles is effectively increased. Besides, a ratio of a pore size of a second through hole 301 to a diameter of a microporous zone 201 is greater than or equal to 0.75 and less than or equal to 1.5, so that the ultrasonic vaporization assembly 20 has a desired noise reduction effect. In addition, the added loading board 23 can reduce a clamping force of a housing 10 and the piezoelectric drive element on the vaporizer plate 22a/22b after the installation of the housing 10 and the ultrasonic vaporization assembly 20, thus effectively reducing vibration performance attenuation caused by the clamping of the vaporizer plate 22a/22b; sealing reliability of the ultrasonic vaporization assembly 20 is increased. Moreover, the second through hole 301 is provided at a position of the loading board 23 corresponding to the microporous zone 201, which can effectively ensure that a liquid inlet surface 221 or a mist outlet surface 222 of the vaporizer plate 22a/22b is connected to the external, so as to ensure that an aerosol substrate or vaporized aerosol particles can pass through smoothly. Furthermore, the embodiments of this application add the loading board 23, which can further change the installation position of the piezoelectric drive element 21 on the vaporizer plate 22a/22b, thus effectively widening the application range of the ultrasonic vaporization assembly 20.

The above description is implementations of this application, and is not intended to limit the patent scope of this application. Any equivalent structure or equivalent process transformations made by using the specification and accompanying drawings of this application, or directly or indirectly applied to other related technological fields are similarly included within the scope of patent protection of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An ultrasonic vaporization assembly, comprising:
a piezoelectric drive element provided with a first through hole;
a vaporizer plate stacked with the piezoelectric drive element, the vaporizer plate comprising a microporous zone at a position corresponding to the first through hole; and
a loading board arranged on a side surface of the vaporizer plate which faces away from the piezoelectric drive element, the loading board being provided with a second through hole at a position corresponding to the microporous zone,
wherein a ratio of a pore size of the second through hole to a diameter of the microporous zone is greater than or equal to 0.75 and less than or equal to 1.5.

2. The ultrasonic vaporization assembly of claim 1, wherein the ratio of the diameter of the microporous zone to the pore size of the second through hole is 1.

3. The ultrasonic vaporization assembly of claim 1, wherein the microporous zone is planar, or
wherein the microporous zone raises towards a mist outlet direction of the ultrasonic vaporization assembly to form a boss.

4. The ultrasonic vaporization assembly of claim 1, wherein the microporous zone is located in a central area of the vaporizer plate.

5. The ultrasonic vaporization assembly of claim 1, wherein the microporous zone comprises a plurality of vaporization through holes, and
wherein a pore size of the vaporization through hole gradually decreases along the mist outlet direction of the ultrasonic vaporization assembly.

6. The ultrasonic vaporization assembly of claim 1, wherein the loading board comprises a metal sheet, and
wherein a thickness of the metal sheet ranges from 0.4 mm to 0.7 mm.

7. The ultrasonic vaporization assembly of claim 1, wherein a ratio of an outer diameter of the vaporizer plate to a thickness of the loading board is between 23 and 40.

8. The ultrasonic vaporization assembly of claim 1, wherein the vaporizer plate has a liquid inlet surface and a mist outlet surface that are arranged opposite each other, the loading board being arranged on the liquid inlet surface, and the piezoelectric drive element being arranged on the mist outlet surface, or
wherein the vaporizer plate has a liquid inlet surface and a mist outlet surface that are arranged opposite each other, the loading board being arranged on the mist outlet surface, and the piezoelectric drive element being arranged on the liquid inlet surface.

9. The ultrasonic vaporization assembly of claim 1, wherein the piezoelectric drive element, the vaporizer plate, and the loading board are all circular and have a same outer diameter,
wherein the vaporizer plate comprises a metallic screen mesh, and
wherein the piezoelectric drive element comprises a piezoelectric ceramic ring.

10. An ultrasonic vaporization device, comprising:
a housing having a liquid storage cavity and a liquid outlet;
the ultrasonic vaporization assembly of claim 1 arranged at the liquid outlet, the ultrasonic vaporization assembly being configured to vaporize an aerosol-forming substrate of the liquid storage cavity when power is turned on; and
a power supply assembly electrically connected to the ultrasonic vaporization assembly, the power supply assembly being configured to supply power to the ultrasonic vaporization assembly.
